(19) United States
(12) Patent Application Publication
(10) Pub. No.: US 2010/0125206 A1
(43) Pub. Date: May 20, 2010

(54) METHODS FOR DIAGNOSIS AND TREATMENT OF VESSEL OCCLUSION

(76) Inventor: Paul David Syme, Roxburghshire (GB)

Correspondence Address: SUGHRUE MION, PLLC, 2100 PENNSYLVANIA AVENUE, N.W., SUITE 800, WASHINGTON, DC, 20037, US

(21) Appl. No.: 12/278,151

(22) PCT Filed: Feb. 2, 2007
(86) PCT No.: PCT/IB2007/000255

§ 371 Date: Dec. 19, 2008

(30) Foreign Application Priority Data
Feb. 2, 2006 (GB) 0602108.3

(51) Int. Cl. A61B 8/06

(52) U.S. Cl. 600/454

(57) ABSTRACT

A method for predicting whether ultrasound insonation is likely to be effective in treating the symptoms of small vessel occlusion, including opening an occluded blood vessel. The method involves the steps of: identifying vessel occlusion in a patient; insonating the appropriate vessel using m-mode ultrasound, identifying on the ultrasound image the area of occlusion, and identifying whether there is blood flow beyond the area of occlusion. Identification of blood flow beyond the area of occlusion is by way of a directional flare signal. This identification indicates that ultrasound insonation is likely to be effective in treating the symptoms of small vessel occlusion, including opening the occluded blood vessel.

The present invention relates to an improved method for diagnosing and treating small vessel disease including small branch arteries of the main intracerebral arteries using ultrasound technology, and in particular m-mode ultrasound.

This invention relates to the subject matter of the Applicant's co-pending International Patent Application No WO04/103184, the contents of which are herein incorporated by reference.

Ultrasound is a medical imaging technique that uses high frequency sound waves to produce images of structures in the body. The technique is based on the pulse-echo principle in which a short burst of ultrasound is emitted from an ultrasound transducer into tissue. When the ultrasound pulse encounters an interface between structures a proportion of the pulse is reflected back towards the transducer. This is often referred to as an echo. These echoes are collected and processed, by timing the period between transmission of the pulse and reception of the echo, in order to generate the image.

Doppler ultrasonography is based upon the Doppler Effect, first described by the Austrian physicist Christian Andreas Doppler in 1842. This effect depends on the principle that the frequency of sound waves reflected from any object remains the same if the object is stationary, but increases or decreases if the object moves towards or away from the sounds of the source respectively. When the object which is reflected in the ultrasound waves is moving, the frequency of the echoes changes, creating a higher frequency when is moving towards the probe, and a lower frequency when it is moving away from the probe. This frequency shift is called the Doppler frequency shift, and forms the basis of Transcranial Doppler ultrasonograhy (TCD).

The use of ultrasound for the detection and treatment of vessel occlusion has previously been proposed in the Applicant's co-pending International Patent Application No WO04/103184. WO04/103184 teaches of “abnormal” ultrasound arterial signals which have been found to be present in ischemic stroke and small branch vessel occlusion and perforator occlusion in general. These high intensity low velocity signals are visible on the ultrasound scan and resemble the short peak systolic wave and diastolic reversal of flow found in circulatory arrest due to brain death. The signal varies from a small triangular noise to a line, although the abnormal signal can also be a bruit and the knock is normally biphasic. Generally the systolic component of the knock can be seen, however a diastolic component is nearly always also observed. The signal repeats with each cardiac cycle, and is generally identifiable at the beginning of systole and at the diacrotic notch. The signals are found in the 300 Hz region of the spectra and have been named “small vessel knock (SVK). They are also found at branches of intracerebral arteries. As disclosed in WO04/103184 these signals are present in small vessel occlusion and intracerebral branch occlusion and transcranial Doppler ultrasonography can therefore be used as a diagnostic tool, even in small vessel occlusion which are generally too small to allow accurate visualisation in CAT or MRI scans.

WO04/103184 also teaches that once occlusion has been diagnosed using this technique, continued insonation by ultrasound at high frequency (typically in the region of 100 Mwatts or above) changes the small vessel knock and branch knock signal. The signal becomes less intense, broadens and a black area appears in the original high intensity signal. The black area often looks like a triangle on the white reflected sound and can be multiple. This has been termed as the insonation window by the inventor, and occurs as there is little or no reflection of the signal back. A low intensity waveform can be seen super-imposed on the high intensity area, often of high velocity as the artery opens. This change is always associated with clinical recovery to some extent. A method of treating the symptoms of small vessel occlusion, for example in the case of stroke, is therefore described.

Different modes of ultrasound are used in medical imaging. Single gate range systems are capable of detecting Doppler signals from one sample volume at a time. In contrast multi-gate (m-mode) systems are able to produce a large number of Doppler signals simultaneously from different selected points along the ultrasound beam. This technology simultaneously measures blood flow and direction at multiple depths, or gates, in contrast to single gate technology which measures activity at one depth or gate. In this respect m-mode Doppler Technology provides better localisation as small vessel knock and branch knock can be seen over larger arterial depths than conventional, single gated transcranial Doppler (TCD) technologies. The present invention is based on the finding that the likelihood of successfully treating the symptoms of small vessel occlusion and branch occlusion with insonation, as described in co-pending WO04/103184, can be successfully predicted using m-mode ultrasound.

According to a first aspect of the present invention there is provided a method for predicting whether ultrasound insonation is likely to be effective in treating the symptoms of small vessel occlusion and branch occlusion comprising the steps of:
(a) identifying vessel occlusion in a patient,
(b) insonating the appropriate vessel using m-mode ultrasound,
(c) identifying on the ultrasound image the area of occlusion,
(d) identifying whether there is blood flow beyond the area of occlusion,

wherein the identification of blood flow beyond the area of occlusion indicates that ultrasound insonation is likely to be effective in treating the symptoms of small vessel occlusion and branch occlusion.

Preferably identification of vessel occlusion in the patient is achieved by transcranial Doppler ultrasonography.

Preferably diagnosis of vessel occlusion is carried out by the identification of abnormal ultrasound arterial signals. The abnormal ultrasound arterial signals are found at the baseline within the +/−300 Hz range.

Typically the abnormal ultrasound arterial signals are associated with each cardiac cycle and have an intensity which varies according to the rhythm of the patient's heartbeat.

In small vessel and branch occlusion the abnormal ultrasound arterial signals typically resemble the short peak systolic wave and diastolic reversal of flow which can be seen with circulatory arrest due to brain death and are high intensity, low velocity signals.

The abnormal ultrasound arterial signals can be seen at the beginning of each systole. The abnormal ultrasound arterial signal may also have a less obvious diastolic component.

Typically the ultrasound frequency is 2 MHz with a filter of less than 300 Hz in order to identify the abnormal ultrasound arterial signals.

Preferably the m-mode ultrasound is configured to be coloured for directionality.

Preferably blood flow beyond the area of occlusion is identifiable by the presence or absence of a flare signal located on the ultrasound image in the area which corresponds to the part of vessel beyond or distal to the area of occlusion represented by the non-coloured area.

Typically the colour of the flare depends on the direction of blood flow within the vessel.

According to a second aspect of the present invention there is provided a method for predicting whether ultrasound insonation is likely to be effective in opening an occluded blood vessel comprising the steps of:
(e) identifying vessel occlusion in a patient,
(f) insonating the appropriate vessel using m-mode ultrasound,
(g) identifying on the ultrasound image the area of occlusion,
(h) identifying whether there is blood flow beyond the area of occlusion,

wherein the identification of blood flow beyond the area of occlusion indicates that ultrasound insonation is likely to be effective in opening the occluded blood vessel.

Preferably identification of vessel occlusion in the patient is achieved by transcranial Doppler ultrasonography.

Preferably diagnosis of vessel occlusion is carried out by the identification of abnormal ultrasound arterial signals. The abnormal ultrasound arterial signals are found at the baseline within the +/−300 Hz range.

Typically the abnormal ultrasound arterial signals are associated with each cardiac cycle and have an intensity which varies according to the rhythm of the patient's heartbeat.

In small vessels including perforators and small branches the abnormal ultrasound arterial signals typically resemble the short peak systolic wave and diastolic reversal of flow which can be seen with circulatory arrest due to brain death and are high intensity, low velocity signals.

The abnormal ultrasound arterial signals can be seen at the beginning of each systole. The abnormal ultrasound arterial signal may also have a less obvious diastolic component.

Typically the ultrasound frequency is 2 MHz or less in order to identify the abnormal ultrasound arterial signals.

Preferably the m-mode ultrasound is configured to be coloured for directionality. The m mode results from several single gated spectra and has a depth intensity and direction.

The area of occlusion is identifiable by the existence of a non-coloured or black area on the ultrasound image of the vessel which corresponds to the area of the occlusion.

Preferably blood flow beyond the area of occlusion is identifiable by the presence or absence of a flare signal located on the ultrasound image in the area which corresponds to the part of vessel beyond or distal to the area of occlusion represented by the non-coloured area. Typically the colour of the flare depends on the direction of blood flow within the vessel.

The present invention will now be described by way of example only, with reference to the accompanying drawings in which:

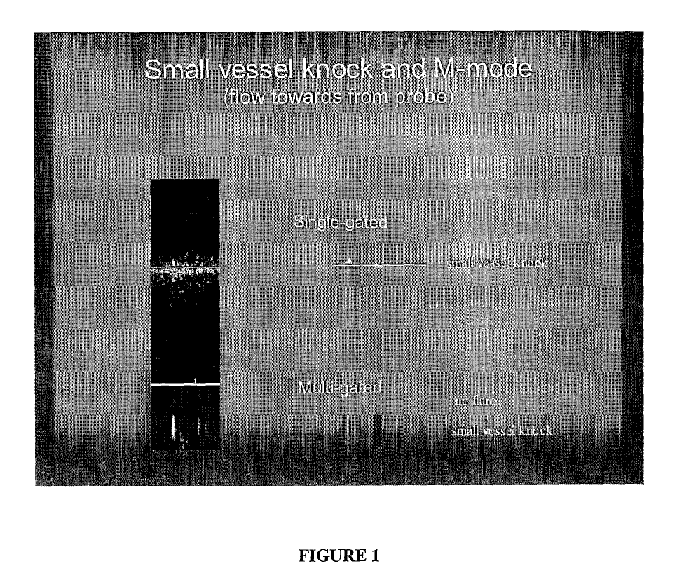

FIG. 1 illustrates the ultrasound image of small vessel knock obtained during insonation with single gated ultrasound (upper part of Figure) and the ultrasound image obtained during insonation with multi gated ultrasound (lower part of Figure), in which further insonation is unlikely to have an effect;

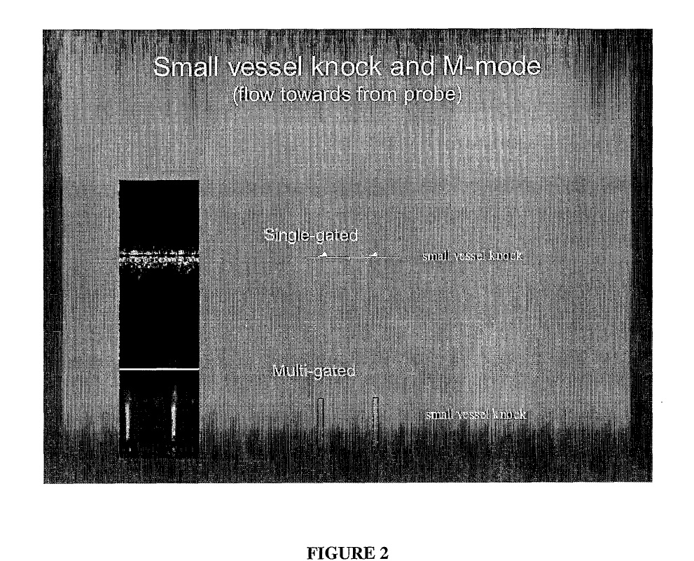

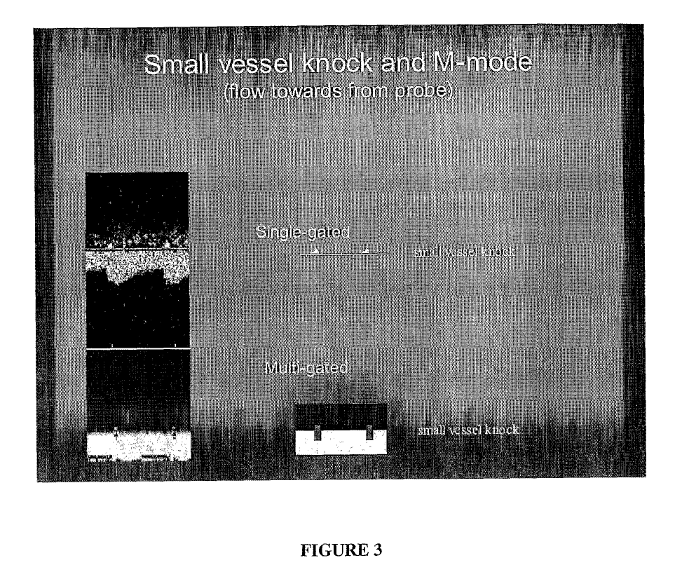

FIGS. 2 and 3 illustrate a lack of flare in a patient with small vessel knock that has been present for at least two years, and;

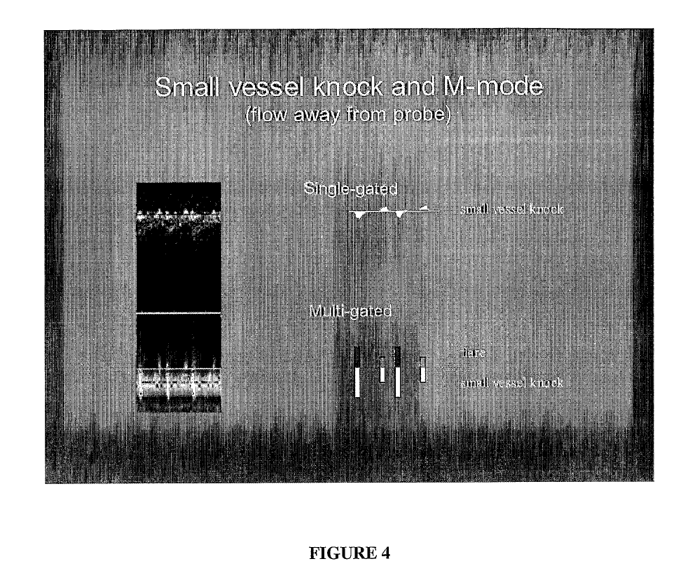

FIG. 4 illustrates the ultrasound image of small vessel knock obtained during insonation with single gated ultrasound (upper image) and the ultrasound image obtained during insonation with multi gated ultrasound (lower image) in which further insonation may be effective.

In the present Application, all references to research are entirely attributable to Paul Syme, who is the inventor.

The inventor's current research indicates that m-mode Doppler ultrasound can be used to predict the likelihood of success in treating the symptoms of small vessel occlusion. Using methods described herein the inventor has identified an m-mode Doppler ultrasound finding that can be used to predict whether insonation treatment will be effective.

Where single gated ultrasound insonation is carried out on an occluded vessel, signals which are normally biphasic, high intensity and low velocity can be identified. The signals are seen at the beginning of systole and at the diacrotic notch, repeat with each cardiac cycle and are found in the 300 Hz region of the spectra. These signals have been named “small vessel knock” (SVK) by the inventor and represent perforator and small branch occlusion. It is the view of the inventor that knock in its various forms represents the true ultrasound finding of arterial occlusion. Knock can be found in all sizes of artery supplying the brain and also in extracranial arteries supplying the brain. Recovery following insonation is dependent on whether following occlusion permanent damage to brain tissue results. The larger the artery the more likely permanent damage will occur. This is linked to abnormal water content in areas of the brain under threat of death following occlusion. These areas are abnormal on an MRI scan when water content of the cells change (MRI positive). Recovery always occurs when the artery opens and the MRI is negative (no abnormal cellular water). When the MRI is positive recovery can still occur but permanent deficits are more likely.

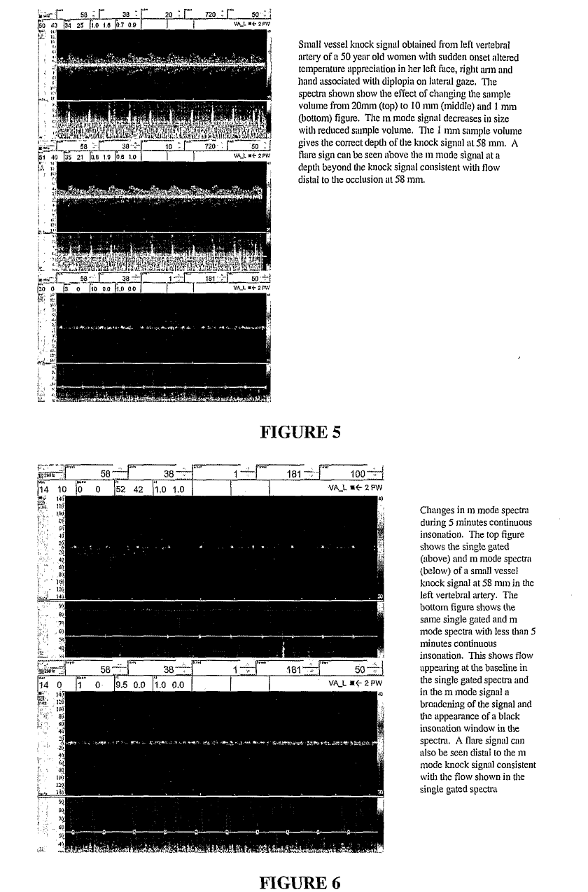

In a number of patients further insonation at high power has been found to change these characteristic signals and results in clinical recovery to some extent. In the present invention it has been found that following identification of small vessel knock m-mode ultrasound technology can be used to predict the likelihood of this further insonation treatment causing clinical recovery.

Where an occluded artery is insonated using m-mode ultrasound the area of small vessel knock, correlating to the site of the occlusion, is shown as a black area. Where this small vessel knock is in an artery flowing towards the probe the m-mode signal is white or red. When small vessel knock is found in an artery flowing away from the ultrasound probe, the small vessel knock signal is inverted and the m-mode ultrasound finding is white or blue. It will be appreciated that although the examples in the present Application refer to particular colours (red and blue) relating to the directionality of blood flow, the actual colours viewed in the ultrasound image may vary depending on the manufacturers choice and the specific configuration of the apparatus. The small vessel knock signal is typically reflected over 10 to 15 millimetres of artery. This depends on the sample volume. To localise the depth of the arterial occlusion the sample volume is reduced as small as possible. FIG. 5. Shows the effect of sample volume on the m mode knock signal ranging from a sample volume of 20 to 1 mm. In m mode changes also occur to the m mode small vessel knock signal during targeted insonation. The signal broadens becomes less intense and a black area can also appear in the centre of the knock signal. This is the m mode insonation window. The appearance of this signal is associated with flow first between the systolic and diacrotic small vessel knock signal on m mode and then in diastole in the single gated spectra. With further opening the m mode small vessel knock signal can disappear and be replaced by a normal m mode aretial flow signal. Recovery occurs when the dark area appears in the m mode small vessel knock signal. These changes are illustrated in FIG. 6.

If the artery is open and there is blood flow beyond the area of occlusion (m-mode small vessel knock) in the artery this is seen as a weaker signal behind the black area of occlusion. The signal in a vessel with small vessel knock with an open artery resembles a candle—with the occluded area at the wick and the open artery beyond as a flame or “flare”. It is likely that this flare is due to clot movement away from the heart during systole and moving back towards the heart at aortic valve closure. The colour seen on the ultrasound image reflects this and also depends on whether the blocked artery is coming towards the probe (red) or away from the probe (blue). The signal could be automatically detected and the sound targeted.

The colour of the flare depends on the direction of flow, being red if flow is towards the ultrasound probe and blue if flow is away from the ultrasound probe. The m-mode ultrasound findings help to explain the small vessel knock signals previously identified in WO04/103184, and are now postulated to aid prediction of the likelihood of the occluded artery re-opening with further insonation.

This is achieved by identifying whether a flare signal is present. In other words, if on the ultrasound image a colour or flare is visible behind the area of occlusion (in other words the area of black), then the possibility that further insonation might be successful in opening the vessel is moderate to high. Alternatively, if no colour is viewed behind the area of the occlusion it is likely that the vessel will not be reopened by further insonation.

The above described findings are illustrated in the Figures. M-mode images were obtained using insonation with m-mode Doppler having 32 sample gates and configured to be coloured red and blue for directionality, with red indicating blood flow towards the ultrasound probe/transducer and blue indicating blood flow away from the ultrasound probe/transducer. In FIG. 1 two distinct areas of blood flow towards the probe are clearly visible in the lower image produced by the m-mode ultrasound and shown schematically on the right. The characteristic high frequency low velocity signal of small vessel knock can be seen at the beginning of systole in the single gated ultrasound image above. No flare is visible in the multi gated image, suggesting further insonation will not be effective in reopening the occluded vessel and treating the symptoms of the vessel occlusion.

FIGS. 2 and 3 illustrate a lack of flare in a patient with small vessel knock that has been present for at least 2 years. As in FIG. 1 two distinct areas of blood flow towards the probe are clearly visible in FIG. 2 in the lower image produced by the m-mode ultrasound and shown schematically on the right. The characteristic high frequency low velocity signal of small vessel knock can be seen at the beginning of systole in the single gated ultrasound image above. In FIG. 3 an area of blood flow towards the probe (red) and away from the probe (blue) are visible in the lower image produced by the m-mode ultrasound.

Referring now to FIG. 4 three distinct areas of blood flow away from the probe are visible (white) in the m-mode image as shown schematically on the right. An area of occlusion represented by a black (non-coloured) are or spot is also visible. However in this case flare is visible beyond or distal to the black area representing the occlusion, suggesting further insonation would be effective in reopening the occluded vessel, and treating the symptoms of the small vessel occlusion. In this case the colour of the flare is blue indicating flow is away from the probe. As the presence of this flare suggests further insonation would assist clinical recovery further insonation at high frequency high frequency (this may be typically in the region of 100 Mwatts or above) is continued. The small vessel arterial knock shown in the single gated images and described above changes during continued insonation. The signal becomes less intense, broadens and a black area appears in the original high intensity signal. The black area often looks like a triangle on the white reflected sound and can be multiple. This has been termed as the insonation window by the inventor, and occurs as there is little or no reflection of the signal back. A low intensity waveform can be seen super-imposed on the high intensity area, often of high velocity as the artery opens. This change is always associated with clinical recovery to some extent. In m mode opening of the artery illustrated in the single gated spectra would result in loss of the intense reflected signal resulting in a red (towards) or blue (away) band corresponding to this artery. Full recovery tends to occur if the vessel fully opens and full opening of the artery tends to result in no recurrence.

Referring now to FIG. 5 the size of the small vessel knock signal is dependent upon the size of the sample volume. This is illustrated for a sample volume of 20, 10, 5 and 1 mm respectively for small vessel knock at an insonation depth of 58 mm in the left vertebral artery of a 50 year old women.

Referring to FIG. 6. Changes to the m mode small vessel knock signal can be seen with increasing duration of insonation. The small vessel knock signal broadens and a black area appears in this m mode signal. This corresponds to changes in the single gated spectra with the appearance of flow between small vessel knock signals. With these changes signal can be seen distal to the small vessel knock signal with a flare signal consistent with some flow entering the artery beyond the knock signal. These changes are associated with recovery.

In the present methods Doppler ultrasound with a frequency of 2 MHz and diameter of 1.6 cm is used. The power for M mode is expressed in mW/cm2. The power used in the method will vary, and for other frequencies will depend on the diameter of the probe head. The power from the probe depends on the pulse repitition frequency (PRF) which also depends on the sample volume and the depth of insonation. For any power, the general rule is that the deeper the signal, the lower the power setting should be. For treatment the power should be maximum, but for safety, used for as short a time as possible. For diagnostic insonation the power should be kept as low as possible.

It follows that the technique should use as low power as possible to identify single-gated and M-mode small vessel knock. Once these signals are identified the parameters should be adjusted to maximise the power. The duration of insonation should be kept as short as possible. In particular the thermal cranial index (TIC=Wₜ/40*d) which is a standard thermal index derived from estimating maximum heating of tissues while accounting for signal attenuation due to cranial bone should be observed, and should be kept below a value of 2.0. In this respect, one or more of the parameters of intensity, sample volume or scale, should be adjusted until the TIC is at or below 2.0. If a TIC value of more than 2.0 cannot be avoided the insonation time should be reduced to 15 minutes. At greater depths the duration of insonation will be greater when targeting deeper SVK signals than for more superficial SVK signals.

The diagnostic and therapeutic method of the present invention can be carried out as in the following example, relevant to stroke:

(a) Identification of the appropriate intracerebral artery is carried out using clinical methods such as assessment of symptoms and knowledge of the vascular anatomy. Abnormal arterial signals (small vessel arterial knock, as described above) are identified using Doppler ultrasound scanning in the appropriate intracerebral artery using visual and audible signals in the manner described above. _{T}hₑ filter is typically reduced to 300 Hz or less so that the signals can be detected around the baseline. Single-gated ultrasound or a combination of single-gated and M mode ultrasound can be used for this. In most cases a combination of both will be used. ie. single gated small vessel knock signals and M mode small vessel knock signals will be looked for at the same time. The depth of the occlusion is determined by reducing the sample volume to a minimum.

(b) Once detected, insonation using m-mode ultrasound is continued to predict the likelihood of the occluded artery re-opening by identification of the presence or absence of a flare signal. If on the ultrasound image a colour or flare is visible behind the black area of occlusion then it is possible that further insonation might be successful in opening the vessel. Alternatively, if no colour is viewed behind the black area of the occlusion it is likely that the vessel will not be reopened by further insonation.

(c) If a flare is visible on the m-mode ultrasound image insonation of the abnormal artery is continued on high power (e.g., often in the region of 100 Mwatts and above) until the artery opens or the systolic triangular signal changes and the insonation window appears for single gated and m mode. If the artery fully opens this is associated with the disappearance of the m-mode SVK signal resulting in a band. Partial opening is associated with broadening of both the single gated and m mode signals with black insonation windows appearing in either signals. Flow can be seen in single gated spectra between the knock signals and or in the diastolic region of the spectra. This change to the single gated spectra is associated with a flare signal in the m mode spectra distal to the m mode knock signal consistent with flow in the artery beyond the obstruction.

This technique is applicable to both ischaemic and haemorrhagic stroke if associated with knock. Patients with the same vessel abnormalities secondary to tumour will also benefit from the above technique. The technique has further applications in other illnesses associated with small to moderate vessel occlusion, such as coronary heart disease, graft rejection, vascular kidney disease, vascular dementia and memory loss and indeed any condition where vessel occlusion can be shown to be linked to that disease.

It should be noted that the embodiments disclosed above are merely exemplary of the invention, which may be embodied in different forms. Therefore details disclosed herein are not to be interpreted as limiting, but merely as a basis for claims and for teaching one skilled in the art as to the various uses of the present invention in any appropriate manner.

1. A method for predicting whether ultrasound insonation is likely to be effective in treating the symptoms of small vessel occlusion, the method comprising the steps of:
(a) identifying vessel occlusion in a patient,
(b) insonating the appropriate vessel using m-mode ultrasound,
(c) identifying on the ultrasound image the area of occlusion, and
(d) identifying whether there is blood flow beyond the area of occlusion, wherein the identification of blood flow beyond the area of occlusion indicates that ultrasound insonation is likely to be effective in treating the symptoms of small vessel occlusion.

2. A method as claimed in claim 1, wherein identification of vessel occlusion in the patient is achieved by transcranial Doppler ultrasonography.

3. A method as claimed in claim 2, wherein diagnosis of vessel occlusion is carried out by the identification of abnormal ultrasound arterial signals.

4. A method as claimed in claim 3, wherein the abnormal ultrasound arterial signals are identified at the signal baseline within the +/−300 Hz range.

5. A method as claimed in claim 3, wherein the abnormal ultrasound arterial signals are associated with each cardiac cycle.

6. A method as claimed in claim 3, wherein the abnormal ultrasound arterial signals have an intensity which varies according to the rhythm of the patient's heartbeat.

7. A method as claimed in claim 3, wherein the abnormal ultrasound arterial signals typically resemble a short peak systolic wave and diastolic reversal of flow which can be seen with circulatory arrest due to brain death.

8. A method as claimed in claim 3, wherein the abnormal ultrasound arterial signals are high intensity, low velocity signals.

9. A method as claimed in claim 3, wherein the abnormal ultrasound arterial signals are identified at the beginning of each systole.

10. A method as claimed in claim 9, wherein the abnormal ultrasound arterial signal comprises a diastolic component.

11. A method as claimed in claim 3, wherein the ultrasound power is reduced to 2 MHz or less in order to identify the abnormal ultrasound arterial signals.

12. A method as claimed in claim 1, wherein the m-mode ultrasound is configured to produce a coloured ultrasound image, said colour being indicative of directionality.

13. A method as claimed in claim 12, wherein the area of occlusion is identifiable as a non-coloured or black area on the ultrasound image of the vessel.

14. A method as claimed in claim 13, wherein blood flow beyond the area of occlusion is identifiable by the presence or absence of a flare signal on the ultrasound image of the vessel.

15. A method as claimed in claim 14, wherein the flare signal is located on the ultrasound image in an area which corresponds to a part of vessel beyond or distal to the area of occlusion.

16. A method as claimed in claim 14, wherein the colour of the flare signal depends on the direction of blood flow within the vessel.

17. A method for predicting whether ultrasound insonation is likely to be effective in opening an occluded blood vessel comprising the steps of:
(a) identifying vessel occlusion in a patient,
(b) insonating the appropriate vessel using m-mode ultrasound,
(c) identifying on the ultrasound image the area of occlusion, and
(d) identifying whether there is blood flow beyond the area of occlusion,
wherein the identification of blood flow beyond the area of occlusion indicates that ultrasound insonation is likely to be effective in opening the occluded blood vessel.

18. A method as claimed in claim 17, wherein identification of vessel occlusion in the patient is achieved by transcranial Doppler ultrasonography.

19. A method as claimed in claim 18, wherein diagnosis of vessel occlusion is carried out by the identification of abnormal ultrasound arterial signals.

20. A method as claimed in claim 19, wherein the abnormal ultrasound arterial signals are identified at the signal baseline within the +/−300 Hz range.

21. A method as claimed in claim 19, wherein the abnormal ultrasound arterial signals are associated with each cardiac cycle.

22. A method as claimed in claim 19, wherein the abnormal ultrasound arterial signals have an intensity which varies according to the rhythm of the patient's heartbeat.

23. A method as claimed in claim 19, wherein the abnormal ultrasound arterial signals typically resemble a short peak systolic wave and diastolic reversal of flow which can be seen with circulatory arrest due to brain death.

24. A method as claimed in claim 19, wherein the abnormal ultrasound arterial signals are high intensity, low velocity signals.

25. A method as claimed in claim 19, wherein the abnormal ultrasound arterial signals are identified at the beginning of each systole.

26. A method as claimed in claim 25, wherein the abnormal ultrasound arterial signal comprises a diastolic component.

27. A method as claimed in claim 19, wherein the ultrasound power is reduced to 2 MHz or less in order to identify the abnormal ultrasound arterial signals.

28. A method as claimed in claim 17, wherein the m-mode ultrasound is configured to produce a coloured ultrasound image, said colour being indicative of directionality.

29. A method as claimed in claim 28, wherein the area of occlusion is identifiable as a non-coloured or black area on the ultrasound image of the vessel.

30. A method as claimed in claim 29, wherein blood flow beyond the area of occlusion is identifiable by the presence or absence of a flare signal on the ultrasound image of the vessel.

31. A method as claimed in claim 30, wherein the flare signal is located on the ultrasound image in an area which corresponds to a part of vessel beyond or distal to the area of occlusion.

32. A method as claimed in claim 30, wherein the colour of the flare signal depends on the direction of blood flow within the vessel.
